(19) United States
(12) Patent Application Publication
(10) Pub. No.: US 2020/0323603 A1
(43) Pub. Date: Oct. 15, 2020

(54) DISPOSABLE GLOVE AND GLOVE DISPENSING APPARATUS

(71) Applicant: GLOVEMATIC PTY LIMITED, Hornsby, NSW (AU)

(72) Inventors: MARK BAYLY, ELTHAM (AU); MICHELLE ROSEVEAR, MOONEE PONDS (AU); LEE BAKER, PRESTON (AU); STEPHAN BACKHAUS, WHITEMARK (AU)

(21) Appl. No.: 16/098,699

(22) PCT Filed: May 3, 2017
(86) PCT No.: PCT/AU2017/050406

§ 371 Date: Nov. 2, 2018

(30) Foreign Application Priority Data
May 3, 2016 (AU) 2016901633

(51) Int. Cl. A61B 42/40; A61B 42/50

(52) U.S. Cl. CPC A61B 42/40; A47G 25/904; A61B 42/50

(57) ABSTRACT

There is provided a package of disposable gloves (10) and a glove dispensing apparatus (60). The package of disposable gloves comprises a plurality of gloves (25) joined to one another in a line wherein adjacent ones of the gloves are connected together by a respective first frangible connection (26) which is breakable to separate the gloves from one another. Each of the gloves has a glove edge (29) lying transversely to the first frangible connection to form an open cuff (30) of each glove (25), and at least one finger (11b) of each glove is connected to an adjacent finger (1b) or thumb (11c) of the glove by a corresponding further frangible connection (52) which is breakable to separate the connected fingers or finger and thumb of the glove. The glove dispensing apparatus (60) can be used for enabling hygienic location of a glove of the glove package (10) on a user's hand. The dispensing apparatus comprises a storage (64, 65) for storing the package of gloves, a glove line feed arrangement (72a, 72b) for advancing the line of gloves to locate an end glove of the line in a hand insertion zone (68) of the apparatus, and a glove opening system including adherent surfaces (83), the glove opening system (70) being operable for contact of the adherent surfaces with opposed first and second layers (18, 20) of the end glove of the line to adhere to the layers and subsequently to draw the adhered layers of the glove apart for allowing a user to insert their hand into the open cuff (30) of the glove in the hand insertion zone (68). There are also provided methods for use of the glove dispensing apparatus.

### FIELD OF THE INVENTION

The present invention relates to a package of disposable gloves and to a glove dispensing apparatus.

### BACKGROUND OF THE INVENTION

The use of disposable gloves is common in many industries, such as food handling establishments where food such as sandwiches or the like may be prepared and sold to a customer, or other open food products such as meats and the like are selected from a tray and wrapped for a customer to purchase. The use of such gloves is intended to improve hygiene and prevent the spread of germs which may take place if such food products are handled by bare hands.

Dispensable gloves are commonly sold in a box. A user pulls the gloves from an opening in the top of the box and subsequently applies them by hand. This requires the user to have significant contact with the gloves prior to, and during, application of the gloves. As such, the outer surface of the glove can become contaminated with germs, dirt and/or other unwanted material present on the user's hands.

An automated glove dispensing machine has been proposed in International Patent Application No. PCT/AU2008/001377 in the name of the present applicant. The machine includes a compartment for storing a roll of dispensable gloves, a moveable indexing arrangement for drawing a line of the gloves from the roll to a hand insertion station of the machine, and a glove opening device for opening the end glove of the line for insertion of a user's hand at the hand insertion station. The roll of gloves comprises a series of panels each comprising a disposable glove wherein consecutive ones of the panels are joined together by a frangible connection. In use, the frangible connection is torn to separate the glove from the line as the user withdraws their hand from the hand insertion station, and the indexing device travels back along the line of gloves to grip the line of gloves at a gripping station of the machine and further draw the line of gloves from the roll with return movement to the hand insertion station.

### SUMMARY OF THE INVENTION

In an aspect of the present invention there is provided a package of disposable gloves, comprising:

a plurality of gloves joined to one another in a line wherein adjacent ones of the gloves are connected together by a respective first frangible connection which is breakable to separate the gloves from one another; and

a glove edge lying transversely to the first frangible connection to form an open cuff of each glove, at least one finger of each said glove being connected to an adjacent finger or thumb of the glove by a corresponding further frangible connection which is breakable to separate the connected fingers or finger and thumb of the glove from one another.

Typically, the connected fingers or finger and thumb of the glove are held immediately next to one another by the further frangible connection.

Typically, the further frangible connection defines at least a portion of an outer edge of each of the connected fingers or finger and thumb of the glove.

Typically, the further frangible connection is breakable to permit freedom of movement of the connected fingers or finger and thumb of the glove relative to one another.

Typically, each finger of the glove is connected to an adjacent finger or thumb of the glove by a respective said further frangible connection.

Typically, the first frangible connection joins the thumb or small finger of the glove to the small finger or thumb of an adjacent glove in the line.

Typically, the first frangible connection joins the thumb of the glove to the small finger of an adjacent upstream glove.

In at least some embodiments the first frangible connection extends from the glove edge to an upper portion of the thumb of the glove and to a lower portion of the small finger of the adjacent glove.

Typically, each glove in the line further comprises a web portion extending from one side of a palm portion of the glove to the first frangible connection connecting the glove to an adjacent upstream glove.

Gloves in the line can also comprise a further web portion extending from an opposite side of the palm portion to the first frangible connection connecting the glove to a downstream glove.

In at least some embodiments, each glove in the line can have one or more machine registrable guides. The machine registrable guides can, for example, comprise at least one guide opening located in a respective web portion of the glove.

In another aspect of the invention there is provided a package of disposable gloves, comprising:

a plurality of gloves joined to one another in a line wherein adjacent ones of the gloves are connected together by a respective first frangible connection which is breakable to separate the gloves from one another; and

a glove edge lying transversely to the first frangible connection to form an open cuff of each glove, and each finger of the glove is connected to an adjacent finger or thumb of the glove by a respective said further frangible connection which is breakable for separation of the connected fingers and the finger and thumb of the glove from one another.

In another aspect of the invention there is provided a disposable glove in which at least one finger of each said glove is connected to an adjacent finger or thumb of the glove by a corresponding frangible connection which is breakable to separate the connected fingers or finger and thumb of the glove from one another.

In another aspect of the invention there is provided a disposable glove in which each finger of the glove is connected to an adjacent finger or thumb of the glove by a respective said further frangible connection which is breakable for separation of the connected fingers and the finger and thumb of the glove from one another. In another aspect there is provided a package of disposable gloves embodied by the invention in combination with a glove dispensing apparatus for dispensing the gloves.

In another aspect of the invention there is provided a dispensing apparatus for enabling hygienic location of a glove on a user's hand, comprising:

a storage for storing a continuous line of gloves wherein adjacent ones of the gloves are connected together by a respective first frangible connection which is breakable to separate the gloves from one another, each glove being formed from first and second layers and having a glove edge lying transversely to the frangible connection to form an open cuff of the glove;

a glove line feed arrangement for advancing the line of gloves to locate an end said glove of the line in a hand insertion zone of the apparatus; and

a glove opening system including adherent surfaces, the glove opening system being operable for contact of the adherent surfaces with the first and second layers of the end glove to adhere to the layers and subsequently to draw the adhered layers of the end glove apart for allowing a user to insert their hand into the open cuff of the glove in the hand insertion zone.

Typically, the glove opening system of the apparatus is located in the hand insertion zone.

Typically, the glove opening system comprises a pair of glove opening elements each having an adherent surface on an inner side thereof, the glove opening system being operable to move at least one of the glove opening elements toward the other of the glove opening elements to effect the contact of the adherent surfaces with a corresponding said layer of the glove whereby the layers of the glove adhere to the adherent surfaces, and then away relative to the other glove opening element to separate the adhered layers and thereby open the glove.

One or both of the glove opening elements can be removable. This allows the glove opening element(s) to be removed for cleaning or to be periodically replaced with new glove opening element(s).

In at least some embodiments, each adherent surface is provided by a coating on the respective glove opening elements.

In other embodiments the adherent surfaces are surfaces of removable gripping portions of the glove opening elements.

Typically, the glove opening elements comprise a pair of arms wherein the arms are disposed opposite to one another and are operable to be moved toward one another for contact of the adherent surfaces with the layers of the glove and subsequently, away from one another to open the glove.

Typically, the glove line feed arrangement is arranged to advance the line of gloves downwardly to the hand insertion zone of the glove dispensing apparatus.

Typically, the glove line feed arrangement comprises a pair of opposed rollers operable to receive the line of gloves, at least one of the rollers being drivable for advancing the line of gloves to locate the end one of the gloves in the hand insertion zone of the apparatus.

Typically, the rollers are arranged to remain in a fixed position within the apparatus as the line of gloves is advanced by operation of the rollers.

Typically, the rollers are separable for allowing the continuous line of gloves to be fed between the rollers prior to operation of the rollers to advance the line of gloves toward the hand insertion zone.

Typically, a dispensing apparatus embodied by the invention further comprises a motor arranged to drive the at least one of the rollers to advance the line of gloves to the hand insertion zone and the apparatus being operable to read an indexing portion of each glove for determining at least one of a speed and period of time that the motor is to be operated.

In at least some embodiments, a glove dispensing apparatus as described herein may further comprise a recognition system for recognising the package of gloves as an authorised package for use in the apparatus, the recognition system comprising an interrogation system for interrogating an information coding device of the package to obtain information from the information coding device for processing of the information to identify the package as an authorised package.

If the package is not provided with an information coding device (e.g., an RFID tag, a QR code or barcode) or the information obtained from the information coding device is not recognised, the package can be determined as not being an authorised (e.g., genuine) package of gloves. In this instance, the apparatus may be configured to not operate with the package (e.g., not dispense gloves from the package).

In another aspect of the invention there is provided a dispensing apparatus for hygienic location of a glove on a user's hand, the apparatus comprising a glove opening system including adherent surfaces, wherein the glove is formed from first and second layers, and the glove opening system is operable for contact of the adherent surfaces with the first and second layers of the glove to adhere to the layers and subsequently to draw the adhered layers of the glove apart to open the glove for allowing a user to insert their hand into the glove.

In another aspect of the invention there is provided a dispensing apparatus for enabling hygienic location of a glove on a user's hand, comprising:

a storage for storing a continuous line of gloves wherein adjacent ones of the gloves are connected together by a respective first frangible connection which is breakable to separate the gloves from one another, each glove being formed from two layers and having a glove edge lying transversely to the frangible connection to form an open cuff of the glove;

a glove line feed arrangement comprising a pair of rollers operable to receive the continuous line of gloves, at least one of the rollers being drivable so as to advance the line of gloves for locating an end one of the gloves in a hand insertion zone of the apparatus; and

a glove opening system operable to separate the two layers of the end glove to allow a user to insert their hand into the open cuff of the glove.

In another aspect of the invention there is provided a method for hygienic location of a glove on a hand, comprising:

loading disposable gloves on to a dispensing apparatus for dispensing of the gloves, each glove being formed from first and second layers, and the apparatus comprising a glove opening system including adherent surfaces, the glove opening system being operable for contact of the adherent surfaces with the first and second layers of a respective said glove to adhere to the layers and subsequently to draw the adhered layers of the glove apart to allow a user to insert their hand into an open cuff of the glove; and

operating the glove opening system of the apparatus whereby the adherent surfaces of the glove opening system adhere to the first and second layers of the glove and draw the layers apart to open the glove for insertion of a user's hand into the glove.

In another aspect of the invention there is provided a glove opening element of a glove dispensing apparatus for dispensing a glove from a package of gloves in which each of the gloves are formed from first and second layers and the gloves in the package are joined together in a line wherein adjacent ones of the gloves are connected by a respective frangible connection which is breakable to separate the gloves from one another, the glove opening element comprising a support with an adherent surface for temporarily adhering to an end said glove of the line in use, and the support being adapted to be fitted to the glove dispensing apparatus for movement into contact with a corresponding said layer of the glove whereby the adherent surface adheres to that glove layer for opening of the glove with relative movement of the support to the other layer of the glove during operation of the glove dispensing apparatus.

In another aspect of the invention there is provided a glove opening element of a glove dispensing apparatus for dispensing a glove from a package of gloves in which each of the gloves is formed from first and second layers and the gloves in the package are joined together in a line wherein adjacent ones of the gloves are connected by a respective frangible connection which is breakable to separate the gloves from one another, wherein the glove opening element comprises a support with an adherent surface for temporarily adhering to an end said glove of the line in use, the support being adapted for being fitted to the glove dispensing apparatus and the adherent surface being located on the support such that the glove opening element is operable with a further glove opening element of the apparatus having a further adherent surface to be moved into contact with the glove whereby each layer of the glove adheres to a respective one of the adherent surfaces of the glove opening elements and the adhered layers are drawn apart upon at least one of the glove opening elements being moved away from the other glove opening element to open the glove with operation of the apparatus, for insertion of a hand of a user into an opened cuff of the glove. Typically, the support of the glove opening element comprises an upper section and a rearwardy directed lower portion extending from the upper section for being disposed alongside the line of gloves and providing the adherent surface.

In at least some embodiments the adherent surface is the surface of an adherent coating applied to the support. In other embodiments the adherent surface may be provided by a gripping portion mounted to the support of the glove opening element. In at least some forms the gripping portion may, for instance, be selected from tapes, strips, panels or sleeves having an adherent coating for contact with the glove as described above.

Hence, in yet another aspect of the invention there is provided a gripping portion adapted to be mounted to a support of a glove opening element of a glove dispensing apparatus for dispensing a glove from a package of gloves in which the gloves are joined together in a line wherein adjacent ones of the gloves are connected by a respective frangible connection which is breakable to separate the gloves from one another, the gripping portion having an adherent surface located for contact with a layer of the glove for temporary adherence of the glove layer to the adherent surface during a glove opening operation of the dispensing apparatus to enable insertion of a user's hand into the opened glove.

In another aspect of the invention there is provided the gripping portion in combination with the support of a glove opening element for a glove dispensing apparatus as described herein.

An apparatus embodied by the present invention for dispensing gloves may also further comprise at least one ultra-violet (UV) light source arranged within a housing of the apparatus for treating the gloves with ultra-violet light.

In another aspect there is provided an apparatus for dispensing gloves, comprising at least one ultra-violet (UV) light source arranged within a housing of the apparatus for treating the gloves with ultra-violet light.

In at least some embodiment, the housing includes a cover, and the UV light source is mounted underneath the cover for irradiating the gloves when the cover of the housing is closed.

Typically, the UV light source is mounted within the housing of the glove dispensing apparatus whereby a hand insertion zone of the dispenser is at least partly shielded from the ultra-violet light in use of the UV light source.

In another aspect of the invention there is provided a method for hygienic location of a glove on a user's hand, comprising:

loading a package of disposable gloves on to a dispensing apparatus for dispensing of the gloves, the package comprising a plurality of gloves joined to one another in a line wherein adjacent ones of the gloves are connected together by a respective first frangible connection which is breakable to separate the gloves from one another, and wherein a glove edge lies transversely to the first frangible connection to form an open cuff of each glove, and at least one finger of each said glove is connected to an adjacent finger or thumb of the glove by a corresponding further frangible connection which is breakable to separate the connected fingers or finger and thumb of the glove from one another, each glove being formed from first and second layers; and

operating the apparatus for dispensing of a glove from the line of gloves, the apparatus comprising a glove line feed arrangement for advancing the line of gloves to locate an end said glove of the line in a hand insertion zone of the apparatus, and a glove opening system including adherent surfaces, the glove opening system being operable for contact of the adherent surfaces with the first and second layers of the end glove to adhere to the layers and subsequently to draw the adhered layers of the glove apart to allow a user to insert their hand into the open cuff of the glove.

In yet another aspect of the invention there is provided a method for hygienic dispensing of a glove, comprising;

providing a package of disposable gloves for being loaded on to a dispensing apparatus for dispensing of the gloves, the package comprising a plurality of gloves joined to one another in a line wherein adjacent ones of the gloves are connected together by a respective first frangible connection which is breakable to separate the gloves from one another, and wherein a glove edge lies transversely to the first frangible connection to form an open cuff of each glove, and at least one finger of each said glove is connected to an adjacent finger or thumb of the glove by a corresponding further frangible connection which is breakable to separate the connected fingers or finger and thumb of the glove from one another, each glove being formed from first and second layers;

loading the package of disposable gloves on to the apparatus; and

operating the apparatus for dispensing of a glove from the line of gloves, the apparatus comprising a glove line feed arrangement for advancing the line of gloves to locate an end said glove of the line in a hand insertion zone of the apparatus, and a glove opening system including adherent surfaces, the glove opening system being operable for contact of the adherent surfaces with the first and second layers of the glove to adhere to the layers and subsequently to draw the adhered layers of the glove apart to allow a user to insert their hand into the open cuff of the glove.

Typically, the package of disposable gloves as described herein is a roll of gloves although the invention is not limited thereto. For instance, a package of disposable gloves may be provided or utilised in a glove dispensing apparatus embodied by the invention in which the gloves are arranged within the package in concertina fashion so as to be folded one next to another for being consecutively drawn from the package of gloves in use.

Moreover, a glove embodied by the invention or dispensed from a dispensing apparatus in accordance with the invention may be a “non-handed” glove that can be worn on either the right or left hand of the use (i.e., the same glove can be worn on either the right or the left hand of the user). Typically, adjacent gloves in a package of disposable gloves as described herein are arranged so that the first frangible connection between the gloves separates the small finger of one glove from the thumb of the other glove. This arrangement is particularly desirable as the user can insert each hand into a respective glove with their hand in a “thumb up” orientation when the gloves are presented by an apparatus embodied by the invention in a corresponding vertical orientation with the opened cuff of the glove facing the user, allowing for ease of donning of the gloves. However, the gloves may be arranged in the line of gloves in other configurations as exemplified below.

By “small finger” of a glove is meant the end finger of the glove for receiving, or which is arranged to receive, the fifth digit of the user's hand (also known as the digitus minimus manus), whether or not that finger of the glove is shorter in length than the other fingers or the thumb of the glove.

Likewise, by the “thumb” of a glove is meant that end finger of the glove for receiving, or which is arranged to receive, the thumb of the user's hand.

Typically, the glove is a loose fitting lightweight “sandwich” type of glove suitable for use by persons working in food related industries or in delicatessens, sandwich bars, confectionary shops or the like where it is necessary to regularly don gloves for short periods when handling food whilst serving a customer and then remove and dispose of them to collect payment from the customer. However, the gloves may be fabricated from various suitable conventionally known materials and may be used for a wide variety of situations or purposes. Advantageously, the opening and presentation of the gloves by embodiments of glove dispensing apparatus as described herein may reduce or prevent contamination of outer surface(s) of the gloves which may otherwise occur when it is necessary for the user to handle the glove with their other bare hand as with the convention donning of gloves. As such, the risk of contamination of the food or whatever is to be handled by the user may also be reduced.

Advantageously also, glove dispensing apparatus in one or more embodiments as described herein may provide for relatively fast donning of gloves, and can avoid the use of single, loose gloves in a box which can otherwise become tangled together resulting in wastage in the event a number of gloves are drawn from the box together. Moreover, apparatus as described herein may be relatively simple in construction and operation.

Other aspects, features, and advantages of the invention will become further apparent from the following detailed description of non-limiting embodiments of the invention when read in conjunction with the accompanying drawings.

### BRIEF DESCRIPTION OF THE ACCOMPANYING DRAWINGS

FIG. 1 shows a plan view illustrating a continuous line of gloves of the prior art;

FIG. 2 is a side view showing of a glove of the line of gloves of FIG. 1;

FIG. 3 is an enlarged plan view of a glove panel of the line of gloves of FIG. 1;

FIG. 4 is a plan view illustrating a continuous line of gloves embodied by the invention;

FIG. 5 is a side view of a package of disposable gloves embodied by the invention in the form of a roll of gloves;

FIG. 6 is a schematic side view illustrating the fabrication of a line of gloves of the invention from first and second glove layers;

FIG. 7 is a front perspective view of a glove dispensing apparatus embodied by the invention; and

FIG. 8 is a view illustrating components of the glove dispensing apparatus of FIG. 7.

### DETAILED DESCRIPTION OF EXEMPLARY EMBODIMENTS OF THE INVENTION

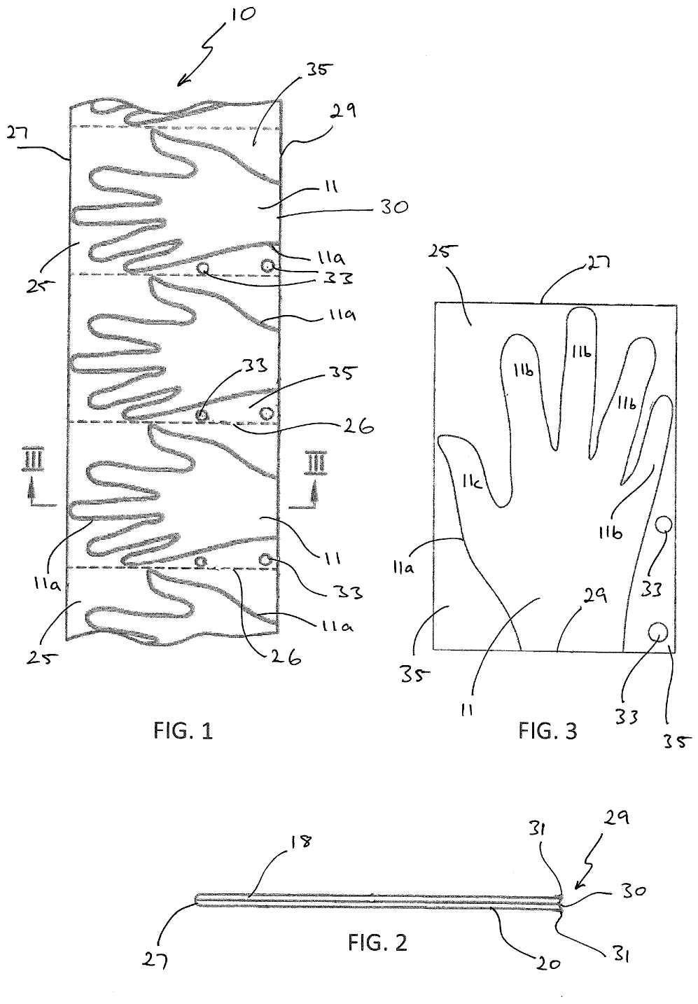

FIG. 1 shows a prior art continuous line of gloves 10. The line of gloves 10 is formed from a first layer 18 and a second layer 20 as illustrated in FIG. 2, which are joined together by a heat seal join that defines the periphery 11a of the hand portion of each glove 11.

The gloves 11 shown in FIG. 1 are in the form of rectangular glove panels 25 (i.e. formed by the two layers 18 and 20) and are connected together by a respective first frangible connection 26. The first frangible connection 26 preferably comprises a perforated tear line so that one glove can be torn from the end the line 10 at a time, so as to separate the glove panels 25.

The glove panels 25 have opposite edges 27 and 29 which are not connected to one another except where the join defining the periphery 11a of the hand portion of each glove intersects or contacts the edges 27 and 29, thereby defining an open cuff 30 of each glove. The edge 29 of the illustrated embodiment is beaded so that the open cuff of each glove 11 has a raised bead 31 along the edge of each layer 18 and 20 of the glove to slightly strengthen that part of the glove.

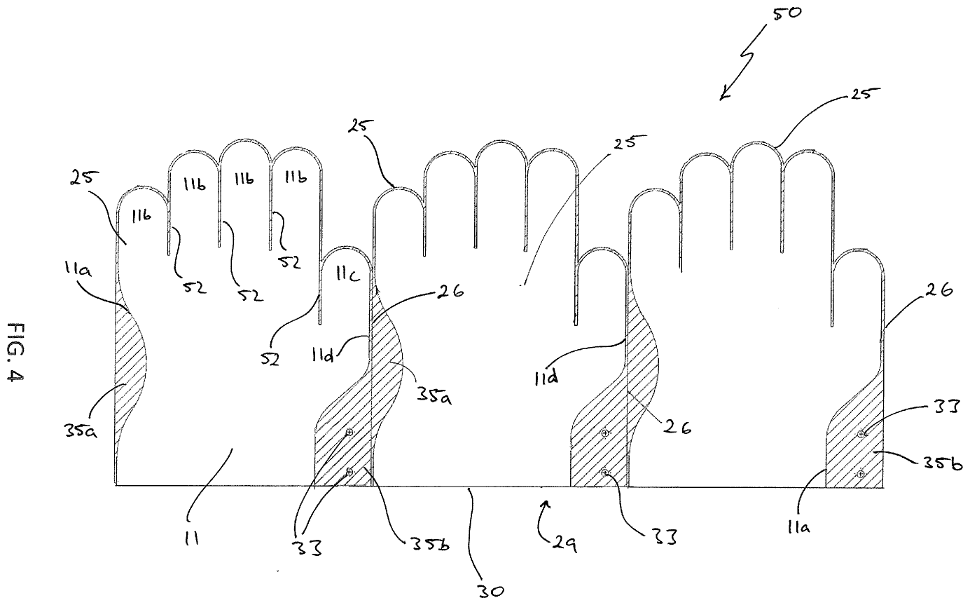

FIG. 3 is an enlarged view of a glove panel 25. As illustrated, webbing 35 formed from the layers 18 and 20 is provided outwardly of the join defining the periphery 11a of the hand portion of each glove. The webbing 35 also separates the fingers and thumb of each glove, and so constitutes a substantial portion of each panel 25. A disadvantage with the glove design depicted in FIG. 1 is that the webs 35 can restrict movement of a user's fingers when in use and further, much of the space taken up by the webbing 35 constitutes wasted material.

Turning now to FIG. 4, there is shown a continuous line of gloves 50 in accordance with an embodiment of the present invention. In the same manner as the prior art line of gloves described above, each glove panel 25 in the line of gloves is formed from two layers 18, 20, with adjacent glove panels 25 being connected together by a respective first frangible connection 26. However, in contrast to the above prior art design, there is no webbing disposed between the fingers 11b and thumb 11c of the glove. Instead, the fingers 11b and thumb 11c of the presently described embodiment are disposed immediately adjacent to one another, and each finger of the glove is connected to an adjacent finger of thumb by a corresponding further frangible connection 52 in the form of a respective perforated tear line. Furthermore, webbing 35a and 35b is only provided to each side of the palm portion 31 of each glove.

As also shown, an outer edge 11d of the thumb 11c of each glove is defined by the corresponding first frangible connection 26 which also defines the outer edge of a lower portion of the small finger 11b of an adjacent upstream glove 11.

A particular feature of the glove depicted in FIG. 4 is that each further frangible connection 52 can be readily broken by the user spreading their hand whereby each of the fingers and thumb 11b, 11c of the glove separate thereby permitting freedom of movement of the fingers and thumb relative to one another. Furthermore, the panel 25 results in a significantly reduced amount of connecting webbing 35a and 35b than the prior art design described above, in turn allowing the size of each panel to be reduced resulting in a substantial saving in material and therefore production costs.

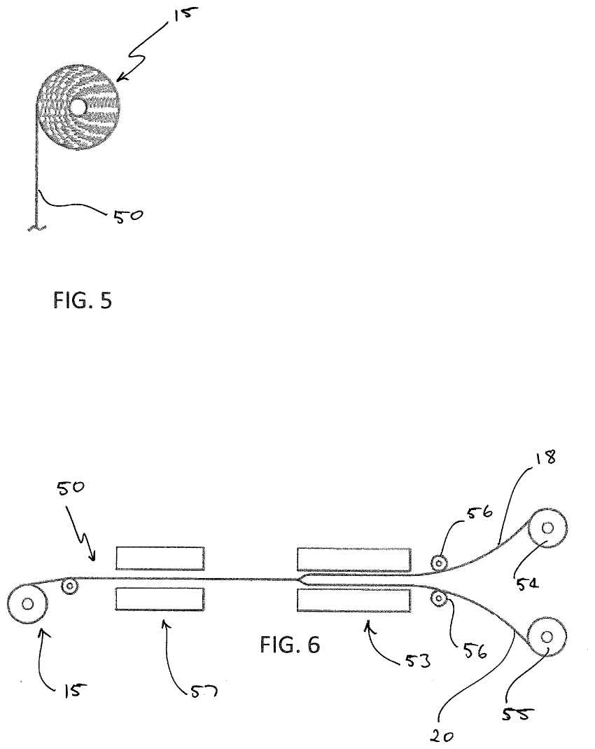

The line of gloves depicted in FIG. 4 may be provided in the form of a package of the gloves, such as a roll 15 as shown in FIG. 5. In other embodiments the package may comprise a layering of the line of gloves one next to another in concertina fashion, or other compact packaging arrangement of the gloves.

Returning to FIG. 4, each panel 25 incorporates an indexing portion in the form of two machine readable registration guides comprising registration holes 33 provided in webbing 35b which are readable by a glove applying apparatus for dispensing of the gloves to determine various machine drive parameters as will be described in more detail below. It will be understood, however, that further markings/indicia and/or other forms of such machine registrable guide(s) may be provided in the line of gloves 50 to facilitate drive parameter recognition by the glove applying apparatus depending on the sensors utilised by the apparatus.

FIG. 6 schematically shows an example of how the package of gloves is formed. More particularly, glove layers 18 and 20 are supplied from supply rolls 54 and 55 of the layer material and are guided around one or more sets of idler rollers 56 to a heat sealing station 53. At the heat sealing station 53 the join defining the periphery 11a of the hand portion of each glove 11 is formed by the application of heat to heat weld the layers 18 and 20 together in a conventional manner. The joined layers 18 and 20 are then indexed to a cutting and perforating station 57 where the respective perforated frangible connections 26 and 52 are formed as are the machine registrable guide holes 33. Alternatively, the join defining the periphery 11a of the hand portion of each glove is formed in the same step or station as the frangible connections 26 and 52 and cutting and removal of any waste layer material.

The formed line of gloves 50 is then rolled onto a roll core (e.g., fabricated from paper or a plastics material) to form the package of gloves 15 shown in FIG. 5. It should be understood that the apparatus shown in FIG. 6 is schematic and so merely illustrative, and is not intended to show all componentry of the machine employed to form the package of gloves, and any suitable system may be utilised.

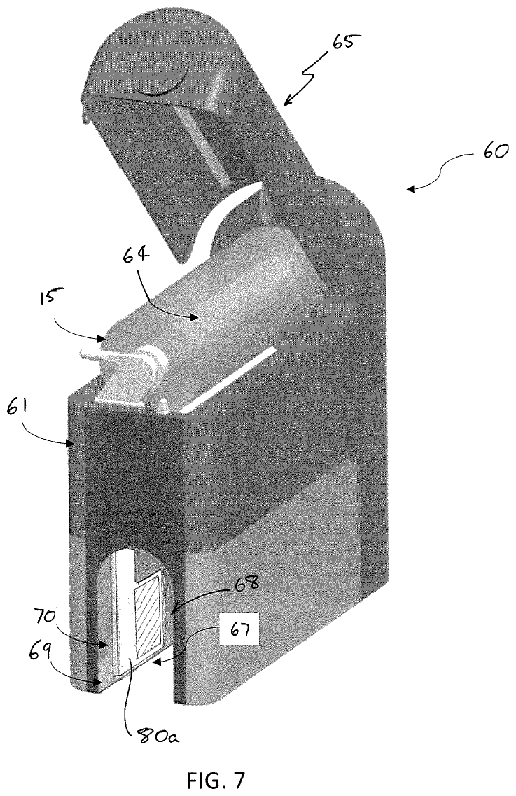

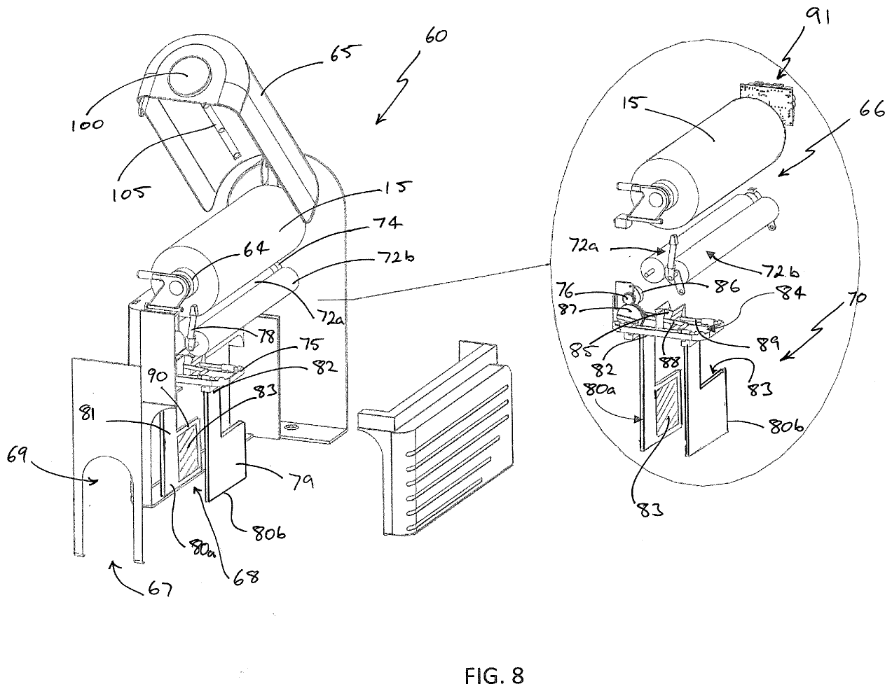

FIGS. 7 and 8 illustrate a glove dispensing apparatus 60 embodied by the invention for the hygienic donning of a glove as described above by a user.

As shown, the apparatus 60 comprises a housing 61 supporting a shaft 64 under a hinged cover 65 of the housing. The shaft 64 in turn receives a roll 15 of the gloves 11. A glove line feed arrangement 66 located within the housing 61 receives and is operable to feed an end glove in the glove line 50 (i.e., packaged as the roll 15) essentially vertically downwardly into a hand insertion zone 68.

A glove opening system comprising a device generally indicated by the numeral 70 is located in the hand insertion zone 68 adjacent a front opening 69 of the housing 61. The glove opening device 70 comprises a pair of spaced apart glove opening elements comprising supports in the form of arms 80a, 80b disposed opposite to one another and which are operable to separate the glove layers of the cuff of the end glove of the roll when the glove is disposed between the arms, for insertion of a user's hand into the glove. Each of the arms 80a and 80b are L-shaped comprising an upright section 81 and a rearwardy directed lower portion 79 extending from the upright section so as to be disposed underneath the shaft 64 and thereby the roll of gloves 15. Thus in use, the line of gloves is hangs between the rearwardly projecting lower portions of the arms for opening of the end glove of the line.

In more detail, and with particular reference to FIG. 8, the glove line feed arrangement 66 comprises a pair of rollers 72a and 72b. Roller 72a is driven by a motor 74 which is coupled to the roller by a reduction gear arrangement in the form of a worm drive comprising a worm gear that is mounted to the rear end of roller 72a and which is driven via a worm screw to which the motor is directly coupled. The motor 74 can be of any suitable type (e.g., an AC, DC or stepper motor) and the roller 72b is free to rotate in situ. A spring loaded lever arrangement 78 allows the rollers 72a and 72b to be levered apart from one another for receiving the glove line therebetween. When returned to the closed position, the rollers 72a and 72b grip the glove line 50 for advancing the line of gloves. In an alternative configuration, the roller 72b may additionally, or alternatively, be driven for advancing the glove line. For instance, where both rollers 72a and 72b are to be driven, an additional gear may be mounted to the end of roller 72b and be meshed with the worm gear driving roller 72a to synchronise rotation of the rollers 72a, 72b so that they rotate in unison with one another but in opposite directions.

As described above, the housing 61 also supports a glove opening device 70 comprising a pair of arms 80a, 80b, and a drive assembly generally indicated by the numeral 84 (see FIG. 8). The arms 80a, 80b are disposed underneath the rollers 72a, 72b in the hand insertion zone 68. In use, the glove line 50 is fed from the roll 15 through a guide generally indicated by the numeral 85 so as to hang between the arms 80a, 80b such that an end glove 11 of the line is located between the lower end portion of each of the arms 80a, 80b. An upper end 82 of each arm 80a, 80b is coupled to a rack 75.

In the embodiment shown, the drive assembly 84 comprises a stepper motor 86 arranged to drive a geared arrangement comprising a cog 76 meshed with a further gear 87 to which are coupled linkages 88 and 89. As best shown in FIG. 8, linkage 88 is connected to arm 80a whilst linkage 89 is coupled to arm 80b so that when the gear 87 is rotated by operation of the stepper motor 86, the linkages 88 and 89 are caused to move back and forth in opposite directions relative to one another to firstly draw the arms 80a and 80b toward one another into contact with the end glove of the line to grip the glove, and then to move the arms away from one another for opening of the glove.

However, it will be understood that any suitable assembly may be utilised for effecting the gripping operation, such as by employing pneumatic pistons coupled to each arm and which respectively extend and retract to move the arms toward and away from each other as described above.

To effect the opening of the end glove of the line, the lower portion of each arm 80a and 80b has an adherent inner surface generally indicated by the numeral 83 for temporarily adhering to the respective outer face of layers 18 and 20 of the end glove of the glove line located therebetween, when the arms are moved into contact with the glove by the drive assembly 84.

In the embodiment shown, the adherent surface is provided by an adherent substance 83 coated directly on the inner face 90 of each arm 80a and 80b. As will be understood, the adherent substance is sufficiently tacky for each of the glove layers to adhere to the corresponding arm 80a or 80b so that the glove layers 18 and 20 are drawn apart whereby the cuff of the glove is opened as the arms are subsequently moved apart from one another by the drive assembly 84.

As the adhesiveness of the afore-mentioned adherent substance coated on the arms may gradually diminish after repeated glove opening operations due to the adherent substance being worn off or as a result of detritus sticking to the surfaces, the arms 80a or 80b can be removed and replaced by new arms 80a and 80b having a fresh coating of the adherent substance on their inner face 90. Alternatively, each of the arms may be periodically removed from the glove dispensing apparatus and washed or otherwise cleaned to remove any unwanted foreign detritus that has accumulated on the adherent surface(s).

In another embodiment, the adherent substance 83 may be coated or otherwise provided on a gripping portion such as an adherent tape, strip or panel or the like that is affixed (e.g., removably mounted) such as by way of a suitable adhesive, Velcro™ hook and loop tape, press studs or other suitable mechanical fastener(s)) to the inner face 90 of the rearwardly directed lower portion of the corresponding arm 80a or 80b. Thus, the tape, strips or panels can be simply detached or otherwise peeled off each arm and be replaced with fresh adherent tape, or fresh adherent strip(s) or panel(s), as the tackiness of the adherent surface(s) decreases over time with use.

In a particularly preferred embodiment, the adherent surface for adhering to a respective layer 18 or 20 of the end glove as described above may be provided by an adherent substance coated on the inner face of a respective sleeve fitted to the rearwardly directed lower portion of each one of the arms 80a or 80b. In this embodiment, each of the sleeves can be simply removed from the arms 80a and 80b and replaced with new sleeves to ensure continued adherence of the glove layers to the arms for opening of the gloves in the line as the gloves are consecutively fed into position for being opened by operation of the arms 80a and 80b. The sleeves may be retained in position on the arms 80a, 80b by virtue of a friction fit on the arms or by any appropriate fastening system such as the provision of Velco™ hook tape strip(s) attached to the interior wall surface of the sleeve so as to be aligned for engagement with loop tape fastened to the respective arms 80a, 80b.

In use, the arms 80a and 80b, or gripping portion(s) (e.g., adhesive tapes, strips, panels or sleeves or the like as described above) will generally remain sufficiently tacky for opening all of the gloves in the line, and may conveniently be removed and replaced at the same time as a new roll of gloves is loaded in the glove dispensing apparatus. In particularly preferred embodiments, replacement gripping portions are provided with each new roll of gloves.

It will be further understood that the adhesiveness of the adherent surfaces of the arms is of a level of tackiness to permit the gripping and opening of the gloves, and to allow the gloves to be readily released from the adherent surfaces as the user withdraws their hand from the apparatus 60, without the material from which the glove is fabricated tearing or more than a comfortable level of force being required for the glove to be peeled from the adherent surfaces as the users withdraws their hand from the apparatus with the glove donned. Some non-limiting examples of suitable adherent substances that may be employed as coatings or to otherwise provide the adherent surfaces on the arms for opening of the gloves include, for instance, commercially available silicon, polyurethane, and epoxy coatings, gels and adhesives (e.g., Silpuran™ 2110 AB silicone gel, Wacker Chemie AG, Munich, Germany), although any suitable known coating e.g., such as coatings conventionally used as an adhesive on removable decals and labels, may also be utilised.

The operation of the dispensing apparatus 60 is as follows. As an initial step, the roll 15 of gloves 11 is slipped onto the shaft 64 to load the roll in the apparatus, and the line of gloves is located between the rollers 72a and 72b as described above prior to closing the hinged cover 65.

A control system generally indicated by the numeral 91 of the dispenser is actuated by operation of a press button or a signal generated by a proximity sensor 100 of the control system that is disposed at the front of the hinged cover of the housing by the user passing their hand over the sensor. The control system also includes an indexing arrangement comprising a further sensor (not shown) that is located internally within the housing 61 which is operable to sense the position of the registration holes 33 of the glove panels to determine the current location of the line and additionally the size of the gloves 11. The sensor can take the form of an optical sensor, proximity sensor or any other suitable sensor for reading spacing and/or size parameter(s) of the registration holes.

With the actuation of the control system the motor 74 is operated causing the driven roller 72a to turn and the line of gloves 11 to thereby be fed essentially vertically downwardly. Further, the internal sensor reads the registration holes 33 for determination by the control system of the size of the gloves and appropriate control (e.g., speed and/or the duration of operation) of the motor 74. Based on the determination by the control system, the driven roller 72a is controlled to stop once the end glove in the glove line 50 is located between the rearwardly projecting lower portions of the respective arms 80a, 80b in the hand insertion zone of the apparatus. In other embodiments, the glove panels may not be provided with machine registable guides (e.g., hole(s), marking(s) or other indicia) and the control system may simply be configured to operate the driven roller for a predetermined time and at a speed for correct location of the end glove in position between the arms 80a and 80b.

The control system then actuates the driving assembly 84 resulting in the operation of the stepper motor 86. The operation of stepper motor 86 causes the arms 80a, 80b to be moved toward each other whereby the adherent surfaces 83 provided on the arms are brought into contact with the respective layers 18 and 20 in the vicinity of the cuff of the end glove 11 whereby the glove layers are gently sandwiched between the arms and adhere to the adherent surfaces on the arms 80a, 80b. With continued operation of the stepper motor 86 during which the motor rotates in the same direction as during the gripping of the glove layers described above, the arms 80a, 80b are moved apart from one another by the linkages 88 and 89 and the adhered glove layers are thereby drawn apart from one another whereby the cuff of the end glove is opened with the return movement of each the arms to its respective initial open position. The glove is therefore held open within the hand insertion zone so that a user can insert his or her hand through front opening 69 of the housing 61 into the glove.

To withdraw the glove, the user simply draws his or her hand downwardly through the open bottom 67 of the housing 61 with a movement in which a short, relatively gentle tug is exerted on the line of gloves to tear the end glove from the line 50 along the perforation provided by the frangible connection 26 that connects the glove 11 to the next upstream glove in the line. Thus, the user's hand is removed from the apparatus with the glove in place on the hand without the user having to touch the exterior of the glove.

To ensure that only the end glove is torn from the line of gloves and that a further length of the glove line is not drawn from the roll during the donning and removal of the end glove, the next upstream glove in the line is gripped between the nip of the rollers 72a and 72b, and roller 72a (and thereby roller 72b) is restrained against rotation. The removal of the users hand with the donned glove can be sensed by a further proximity sensor which provides a signal to the control system to operate the apparatus 60 to feed and open another glove ready for the user to insert his or her other hand into the glove. In other embodiments, however, the control system will generally be configured such that the press button or proximity sensor 100 needs to be manually pressed or operated by the user to cause a new glove to be located and opened in the hand insertion zone of the apparatus as described above for donning of the glove by the user.

Further, one or both of the arms 80a and 80b can be respectively hingedly connected to the rack 75 or other support surface(s) of the dispenser so as to be biased toward the other arm into a normal upright resting position by biasing means (e.g., suitable spring arrangement(s)), to allow a degree of relative movement of the arms away from the other in the event the user bumps one of the arms apart or the arms are otherwise forced apart as the user inserts their hand into the opened glove.

The gloves are generally oversized for ease of insertion of the user's hand into the gloves once they have been opened. Usually the gloves will be lightweight sandwich gloves of the type commonly used in sandwich bars, delicatessens and like food outlets involving the handling and preparation of food for a customer and which are only worn for a short time before being removed and discarded before the next customer is served. However, any suitable glove type whereby the layers of the glove can adhere to, and be drawn apart, for donning of the glove as described herein may be utilised. The gloves may, for example, be polyethylene (PE), vinyl (polyvinyl chloride), nitrile (carboxylated butadiene-acrylonitrile) or polyester gloves. Typically, the gloves with be selected from lightweight, polyethylene (PE) gloves.

As the adherent surfaces on the arms 80a and 80b contact the end glove 11 in the vicinity of the cuff of the glove, the risk of contamination of the hygienic working surfaces of the glove by the adherent substance utilised is minimised and/or avoided.

To further enhance hygiene, at least some glove dispensers in accordance with the invention may further include one or more ultraviolet (UV) light sources provided within the housing for surface treatment of the gloves with sterilizing UV light (e.g., UV light in a wavelength range of from 200 nm to 280 nm and preferably, in the range of from 250 nm-280 nm). The UV light source(s) may, for instance, be selected from UV lamp(s) or UV light emitting diodes (e.g., UV LED strips including multiple spaced apart UV emitting LED's). In the illustrated embodiment, a UV LED strip 105 is shown mounted on the underside of the hinged cover 65 facing the roll of gloves 15.

For safety reasons, the UV light source(s) is/are typically switched off during the opening of the end glove of the glove line in the hand insertion zone and whilst the user's hand is located in the hand insertion zone. Desirably also, the UV light source(s) is/are located within the housing 61 such that the hand insertion zone is at least partly shielded from the emitted UV light e.g., by the roll of gloves. Likewise, the UV light source(s) can only operate when the hinged cover is closed. Further, in at least some embodiments, the duration of operation of the UV light source(s) is controlled by the control system, and may only be switched back on by the control system after a predetermined period of time following detection of the withdrawal of the users hand from the hand insertion zone by a proximity sensor as described above. However, any safe mode of operation of the UV light source(s) can be employed.

To reduce or minimise surface charge build up on the line of gloves and the resulting potential for unwanted electrostatic attraction of the line to one or other of the arms 80a or 80b or other components of the dispensing apparatus 60 as the line of gloves is fed downwardly into the hand insertion zone of the apparatus, the roll of gloves may be grounded to dissipate charge on the line.

Whilst the illustrated glove dispensing apparatus is configured such that the line of gloves is fed essentially vertically downwardly from the glove roll to the hand insertion zone other embodiments may be provided in which the line of gloves is fed from the roll 15 in a different direction and presented in the hand insertion zone in, for example, a substantially horizontal orientation. Any suitable such glove feed arrangement for feeding of the gloves in this manner may be utilised. As will be understood, in this instance, the opposed arms 80a and 80b are also oriented horizontally for being sequentially moved toward and away from each other to open the end glove of the line in use. Further, rather than the thumb of each glove 11 in the line of gloves 50 being connected to the small finger of the adjacent upstream glove by a respective frangible connection 26 as shown in FIG. 4, gloves in consecutive pairs may be joined “thumb to thumb” by a frangible connection 26 whereby the thumbs of each glove pair are side by side and the second glove in each pair is connected to the first glove in the next upstream glove pair in a “thumb to small finger” configuration by a corresponding frangible connection 26. Other alternative arrangements of gloves in the line are also possible (e.g., wherein respective pairs of the gloves in the line are in a “small finger to small finger” configuration) and all such embodiments are expressly encompassed by the invention.

Typically, however, each respective first frangible connection 26 in a line of gloves will join the thumb or small finger of one said glove to the small finger or thumb of an adjacent glove in the line in embodiments of the invention. Thus, each respective frangible connection typically extends from the glove edge 29 to an upper portion of the thumb or lower portion of the small finger of one glove in the line and to a lower portion of the small finger or upper portion of the thumb of an adjacent glove.

Moreover, whilst the arms 80a and 80b of the dispenser apparatus exemplified above are L-shaped, it will be understood that arms of various other shapes may also be utilised.

The control system of a glove dispensing apparatus embodied by the invention may also include a recognition system configured to recognise whether the package of gloves 15 is a genuine, authorised package (i.e., an OEM package) for use in the dispensing apparatus, or a non-genuine aftermarket package. In a preferred embodiment, the apparatus may only become operational after recognising the package as being a genuine package. According to such a configuration, in one for more forms, the control system 91 implements a processor which is communicable with an interrogation system operable to interrogate an information encoding device located on the package. Herein the term “processor” is used to refer generically to any device that can process instructions based on programmed code and may include: a microprocessor, microcontroller, programmable logic device or other computational device, a general-purpose computer (e.g., a PC) or a server.

In a particular embodiment, the interrogation system comprises an RFID reader that is controlled to read an information encoding device in the form of an RFID tag (e.g., an RFID smart label) located on the package (e.g., affixed by a suitable adhesive to the roll core of the package). The RFID tag may be encoded with any desired information that can be read by the RFID reader and subsequently interpreted by the processor as being representative of a genuine package of gloves 15. In an alternative embodiment, the information encoding device may comprise a QR code or barcode disposed on the package and readable by an image scanner implemented by the processor. For example, the QR code or barcode may be printed on respective of the gloves, or a label bearing the QR code or barcode may be affixed to at least a leading one of the gloves in a position to be read by the image scanner as the line of gloves is fed into position for dispensing of the gloves in use. However, any suitable arrangement or location of the information encoding device on the package of gloves is expressly encompassed. It will also be understood that the recognition system and information encoding device should not be seen as being limited to those forms described above and may comprise any suitable electronic or electro-mechanical interrogation arrangement.

Additionally, the control system 91 may further include suitable communications hardware and software for communicating with a remote system (e.g., via the Internet) for determining whether the information extracted from the information encoding device is associated with a genuine package of gloves. The remote system may, for example, implement a server computer system for receiving and sending communications to the control system 91.

Further, the control system 91 may be operable, under the control of the processor, to communicate with the remote system for glove ordering purposes. For example, the processor may be communicable with a sensor that detects and outputs a signal when a new package of gloves has been inserted into the dispenser. Responsive to detecting the sensor signal, the processor may cause the control system 91 to send a communication to the remote system to indicate that a new package of gloves has been inserted into the dispenser. Responsive to receiving the communication, the remote system generates an order for dispatching one or more new packages of the gloves to an address registered for the corresponding glove dispensing apparatus (e.g., identified by an address or other suitable identification information sent with the communication). In at least some embodiments, for instance, the remote system may count, or otherwise effect the recording of, the number of glove packages inserted into the dispenser over a period of time (e.g., days or weeks), and place an order for dispatch of one or more new glove packages (e.g., one or more boxes of new glove packages) for timely delivery to the registered address to ensure the registered user of the dispenser does not run out of the disposable gloves and has sufficient package(s) of the gloves available to them for their needs.

From the above, it will be understood that at least some embodiments of gloves and/or dispensing apparatus in accordance with the invention provide one or more of the following advantages.
The connection of the gloves together by a respective frangible connection 26 allows the gloves to be readily provided in the form of a roll or other suitable package form ready for use.
The connection of finger(s) and/or thumb of the gloves by the further frangible connection(s) 52 allows the gloves to be readily donned by the user once they have been opened.
The gloves can be rapidly and conveniently presented in an opened condition for donning by the user.
The provision of the gloves in a neat package as described herein avoids multiple gloves being drawn from a box at the same time as in the prior art.
The gloves can be opened and donned without the user touching the exterior of the gloves with their bare hands, thereby reducing the risk of contamination of the gloves and enhancing hygiene.
The package of gloves can be quickly loaded into the glove opening apparatus.
The arms 80a and 80b and/or where utilised, the tape, strip, panel or sleeve providing the adherent surface(s) of the arms, are readily removable for easy and rapid replacement, and can be replaced at the same time as the glove dispensing apparatus is loaded with a new package of gloves.
The glove opening system provides an elegant solution for opening of the gloves with a relatively low number of moveable parts.

In the foregoing description of preferred embodiments, specific terminology has been used for the sake of clarity. However, the invention is not intended to be limited to the specific terms so selected, and it is to be understood that each specific term includes all technical equivalents which operate in a similar manner to accomplish a similar technical purpose. Terms such as “front” and “rear”, “inner” and “outer”, “above”, “below”, “upper” and “lower” and the like are used as words of convenience to provide reference points and are not to be construed as limiting terms.

The reference in this specification to any prior publication or information is not, and should not be taken as, an acknowledgement or admission or any form of suggestion that the prior publication or information forms part of the common general knowledge in the field of endeavor to which this specification relates.

In this specification, the word “comprising” is to be understood in its “open” sense, that is, in the sense of “including”, and is thus not limited to its “closed” sense, that is the sense of “consisting only of”. A corresponding meaning is to be attributed to the related words “comprise”, “comprised” and “comprises” where they appear.

The foregoing describes exemplary embodiments of the invention(s) and it will be understood that alterations, modifications, additions and/or changes can be made thereto without departing from the scope of the invention. The described embodiments are therefore, only illustrative and not restrictive. Accordingly, the various embodiments described above may be implemented in conjunction with other features or embodiments. For example, aspects of one embodiment may be combined with aspects of another embodiment to realize yet additional embodiments of the invention.

1. A package of disposable gloves, comprising:
a plurality of gloves joined to one another in a line wherein adjacent ones of the gloves are connected together by a respective first frangible connection which is breakable to separate the gloves from one another;
a glove edge lying transversely to the first frangible connection to form an open cuff of each glove, at least one finger of each said glove being connected to an adjacent finger or thumb of the glove by a corresponding further frangible connection which is breakable to separate the connected fingers or finger and thumb of the glove from one another.

2. A package according to claim 1, wherein the connected fingers or finger and thumb of the glove are held immediately next to one another by the further frangible connection.

3-4. (canceled)

5. A package according to claim 1, wherein each finger of the glove is connected to an adjacent finger or thumb of the glove by a respective said further frangible connection.

6-12. (canceled)

13. A package of disposable gloves according to claim 1 in combination with a glove dispensing apparatus for dispensing the gloves.

14. A disposable glove in which at least one finger of each said glove is connected to an adjacent finger or thumb of the glove by a corresponding frangible connection which is breakable to separate the connected fingers or finger and thumb of the glove from one another.

15-18. (canceled)

19. A dispensing apparatus for enabling hygienic location of a glove on a user's hand, comprising:
a storage for storing a continuous line of gloves wherein adjacent ones of the gloves are connected together by a respective first frangible connection which is breakable to separate the gloves from one another, each glove being formed from first and second layers and having a glove edge lying transversely to the frangible connection to form an open cuff of the glove;
a glove line feed arrangement for advancing the line of gloves to locate an end said glove of the line in a hand insertion zone of the apparatus; and
a glove opening system including adherent surfaces, the glove opening system being operable for contact of the adherent surfaces with the first and second layers of the end glove to adhere to the layers and subsequently to draw the adhered layers of the glove apart for allowing a user to insert their hand into the open cuff of the glove in the hand insertion zone.

20. (canceled)

21. An apparatus according to claim 19, wherein the glove opening system comprises a pair of glove opening elements each having an adherent surface on an inner side thereof, the glove opening system being operable to move at least one of the glove opening elements toward the other of the glove opening elements to effect the contact of the adherent surfaces with a corresponding said layer of the glove whereby the layers of the glove adhere to the adherent surfaces, and then away relative to the other glove opening element to separate the adhered layers and thereby open the glove.

22-24. (canceled)

25. An apparatus according to claim 21, wherein the glove opening elements comprise a pair of arms, the arms being disposed opposite to one another and are operable to be moved toward one another for contact of the adherent surfaces with the layers of the glove, and away from one another to open the glove.

26. An apparatus according to claim 25, wherein the adherent surfaces are removable from the arms.

27-34. (canceled)

35. A dispensing apparatus for hygienic location of a glove on a user's hand, the apparatus comprising a glove opening system including adherent surfaces, wherein the glove is formed from first and second layers, and the glove opening system is operable for contact of the adherent surfaces with the first and second layers of the glove to adhere to the layers and subsequently to draw the adhered layers of the glove apart to open the glove for allowing a user to insert their hand into the glove.

36. A dispensing apparatus for enabling hygienic location of a glove on a user's hand, comprising:
a storage for storing a continuous line of gloves wherein adjacent ones of the gloves are connected together by a respective first frangible connection which is breakable to separate the gloves from one another, each glove being formed from two layers and having a glove edge lying transversely to the frangible connection to form an open cuff of the glove;
a glove line feed arrangement comprising a pair of rollers operable to receive the continuous line of gloves, at least one of the rollers being drivable so as to advance the line of gloves for locating an end one of the gloves in a hand insertion zone of the apparatus; and
a glove opening system operable to separate the two layers of the end glove to thereby allow a user to insert their hand into the open cuff of the glove.

37-43. (canceled)
